(19) United States
(12) Patent Application Publication
(10) Pub. No.: US 2008/0112912 A1
(43) Pub. Date: May 15, 2008

(54) Composition For Hair Care

(76) Inventors: Christian Springob, Lorsch (DE); Timothy Mueller, Schaafheim (DE); Ursula Hehner, Brensbach (DE)

Correspondence Address: MICHAEL J. STRIKER, 103 EAST NECK ROAD, HUNTINGTON, NY, 11743, US

(21) Appl. No.: 11/574,685

(22) PCT Filed: Sep. 9, 2005
(86) PCT No.: PCT/EP05/09709

§ 371 Date: Dec. 17, 2007

(30) Foreign Application Priority Data
Sep. 9, 2004 (DE) 10 2004 044 005.0

(51) Int. Cl. A61K 8/89

(52) U.S. Cl. 424/70.12

(57) ABSTRACT

The present invention relates to a composition containing
a) an organo-modified silicone;
b) if necessary, a C₃-C₂₂ alcohol;
c) if necessary, a cationic surfactant;
d) if necessary, a volatile silicone oil;
e) at least 30 wt % ethanol, in relation to the total weight of the composition; as well as
f) if necessary, additional additives that are different from components a to e,
   wherein the total quantity of components a to f is 100 wt %. The invention also relates to a process for producing a composition, the composition obtainable as a result of this process, a process to treat hair, the use of a composition to treat hair, as well as the use of a composition to produce an agent to treat hair.

The present invention relates to a composition, a process for producing a composition, the composition obtainable as a result of this process, a process to treat hair, the use of a composition to treat hair, as well as the use of a composition to produce an agent to treat hair.

The physical, chemical, and morphological properties of hair are negatively influenced by different types of effects. Thus, the hair is greatly damaged by cosmetic treatments such as repeated bleaching, permanent waves, and coloring, but also by frequent washing of the hair with degreasing surfactants, by climate influences such as differences in humidity and temperature, or the intensive effect of sunlight, as well as by mechanical treatments such as brushing, combing, and towel-drying. The hair becomes brittle and loses its shine. A silky shine, low level of porosity, and a pleasant smooth feel (“good to touch”) are considered the features of natural, healthy hair.

Agents are already known that protect and care for hair or return the shine to it. These agents can be rinsed out of the hair after the effect has been achieved (so-called rinse-off products) or they can also be left on the hair (so-called leave-on products).

The agents used for hair care can be formulated so that they are not just used to care for and protect the individual hairs but also generally improve the appearance of the hairstyle, for example, by leaving the hair sleek and thus easy to style, provide the hair a pleasant feel, and make it silky smooth. These types of preparations are also commonly characterized as hair conditioners or hair-repair products. Compositions that are suitable for this purpose are described, among other things, in the monograph by K. Schrader, Fundamentals and Formulations of Cosmetics, 2nd edition, (Hüthig Buch Verlag [publishing company], 1989) on pages 722 to 781 under the keyword “hair post-treatment agents”.

Hair care products such as hair rinses or hair-repair agents are, for example, also known from WO 98/43599 A1 and DE 197 03 475 A1.

The hair care products described in this prior art are in the form of creamy emulsions or dispersions and consist of an aqueous, hydrophilic phase and an oil- or wax-containing hydrophobic phase. These conventional emulsion- or dispersion-type hair care products have the disadvantage, however, that they are difficult to distribute on the hair. In addition, these emulsion- or dispersion-type hair care products are often heavy, fatty, and greasy. Furthermore, the hair care products based on emulsions or dispersions often cause a disadvantageous reduction in the volume of the dry hair due to the waxes that are typically contained in these types of products.

Thus, the object of the present invention was to reduce or overcome altogether the disadvantages arising from the prior art, which are associated with conventional hair care products.

Particularly, the object of the present invention was to provide a composition that can be used as a hair care agent that would be easier to distribute in the hair in comparison to conventional hair care agents and would also simultaneously exhibit an intensive hair care effect that could be compared with a hair rinse or hair-repair product.

An additional object of the present invention was to provide a composition that could be used as a hair care agent, which would feel less heavy, fatty, and greasy than the emulsion- or dispersion-type hair care products known from the prior art. In addition, this hair care agent was also intended to cause no or at a minimum less reduction in the volume of the dry hair as compared to conventional hair care agents.

Another object of the present invention was to provide a process that could be used to produce the compositions for use as hair care agents with the aforementioned advantages with respect to conventional hair care agents.

Furthermore, an object of the present invention was to provide a process with which hair could be cosmetically treated in the easiest manner possible.

The technical solutions described in the following category-forming claims contribute to achieving these objects, wherein the claims dependent on the category-forming claims represent advantageous embodiments of this solution.

A composition according to the invention, particularly as a hair care agent, which is preferably present in the form of a liquid, contains the following components:
a) an organo-modified silicone;
b) if necessary, a C₃-C₂₂ alcohol preferably different from component e, wherein the alcohol can be contained in the composition preferably in a quantity in a range of from 0.01 to 15 wt %, preferably in a range of from 0.025 to 10 wt %, even more preferably in a range of from 0.05 to 5 wt %, and most preferably in a range of from 0.1 to 2 wt %, based on the total weight of the composition in each case;
c) if necessary, a cationic surfactant;
d) if necessary, a volatile silicone oil;
e) at least 30 wt %, preferably at least 40 wt %, preferably more than 47.5 wt %, especially preferably at least 50 wt %, and further preferably at least 60 wt %, based on the total weight of the composition in each case, ethanol or isopropanol, preferably ethanol; as well as
f) if necessary, additional additives that are different from components a to e,
   wherein the total quantity of components a to f is 100 wt %.

In a preferred embodiment of the composition according to the invention, it also contains the following in addition to component e:
0.01 to 15 wt %, preferably 0.025 to 10 wt %, even more preferably 0.05 to 5 wt %, and most preferably 0.1 to 2 wt % of component a;
0.01 to 40 wt %, preferably 0.025 to 30 wt %, even more preferably 0.05 to 20 wt %, and most preferably 0.1 to 10 wt % of component b;
0.01 to 15 wt %, preferably 0.025 to 10 wt %, even more preferably 0.05 to 5 wt %, and most preferably 0.1 to 2 wt % of component c;
0.01 to 40 wt %, preferably 0.025 to 30 wt %, even more preferably 0.05 to 20 wt %, and most preferably 0.1 to 10 wt % of component d; as well as
0.01 to 15 wt %, preferably 0.025 to 10 wt %, even more preferably 0.05 to 5 wt %, and most preferably 0.1 to 2 wt % of component f, with all quantities given based on the total weight of the composition in each case.

In an additional, particular embodiment of the composition according to the invention, it is anhydrous or contains no more than 20 wt %, preferably no more than 15 wt %, particular preferably no more than 10 wt %, and most preferably no more than 5 wt % water, based on the total weight of the composition in each case. The hair care agent according to the invention is suitable for manual distribution and, in particular, massaging into the hair, with the exception of a hairspray.

### Organo-Modified Silicone (Component A)

“Organo-modified silicone” in terms of the present invention is understood to mean a polysiloxane, with which at least one part of the groups bound to the Si—O—Si backbone has atoms, which are different from carbon and hydrogen and preferably have nitrogen. The groups bound to the backbone preferably represent branching of the backbone, wherein the branching has a lower molecular weight than the chain forming the backbone has.

The organo-modified silicone contained as component a in the composition according to the invention preferably has an average weight of the molecular weight, preferably determined by gel permeation chromatography, ranging from 100 to 1,000,000 g/mol, particularly preferably ranging from 250 to 500,000 g/mol, further preferably ranging from 500 to 100,000 g/mol and most preferably in a range of from 1,000 to 50,000 g/mol.

If the organo-modified silicone is a nitrogen-containing silicone, it is further preferable according to the invention if the nitrogen-containing silicone is an amino-functional silicone, which preferably has an amine portion (meq/g), preferably determined by titration with perchloric acid, in a range of from 0.01 to 3.0, especially preferably in a range of from 0.025 to 2.75, further preferably in a range of from 0.05 to 2.3, and most preferably in a range of from 0.1 to 0.5.

“Amino-functional silicone” in terms of the present invention is understood to be silicone having at least one primary, secondary, or tertiary amino group (=amine-substituted silicone) or, however, at least one quaternary ammonium group (=cationic silicone).

Furthermore, is it is preferable according to the invention if the organo-modified silicone is a nonvolatile silicone, which has a vapor pressure of preferably less than 10 mbar, especially preferably less than 1 mbar, even more preferably less than 0.5 mbar, further preferably less than 0.1 mbar, and most preferably less than 0.05 mbar at 21° C.

In an especially preferred embodiment of the composition according to the invention, the organo-modified silicone is a linear, preferably nonvolatile, amino-functional silicones of Structure I,

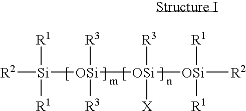

or a linear, preferably nonvolatile amino-functional silicone of Structure II

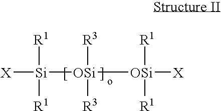

or a cyclic, preferably nonvolatile amino-functional silicone of Structure III

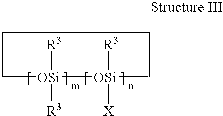

or a mixture of at least two structurally different amino-functional silicones, each of which can be described by structures I, II, or III,
wherein
the R1 groups within the structures, independently from one another, can be the same or different and represent a hydrogen atom, a C₁ to C₁₀ alkyl group, preferably a C₁ to C₅ alkyl group, and further preferably a C₁ to C₄ alkyl group, wherein the methyl group is most preferred, a radical containing aryl groups, preferably a benzyl group or a phenyl group, a hydroxy group, a C₁ to C₁₀ alkoxy group, or a C₂ to C₁₀ acetoxy group, preferably a C₁ to C₅ alkoxy or a C₂ to C₅ acetoxy group, and further preferably a C₁ to C₃ alkoxy or a C₂ to C₃ acetoxy group;
the R² groups within the structures, independently from one another, can be the same or different and represent a hydrogen atom, a C₁ to C₁₀ alkyl group, preferably a C₁ to C₅ alkyl group and further preferably a C₁ to C₄ alkyl group, wherein the methyl group is most preferred, a radical containing aryl groups, preferably a benzyl group or a phenyl group, a hydroxy group, a C₁ to C₁₀ alkoxy group, or a C₂ to C₁₀ acetoxy group, preferably a C₁ to C₅ alkoxy or a C₂ to C₅ acetoxy group, and further preferably a C₁ to C₃ alkoxy or a C₂ to C₃ acetoxy group, or a —(CH₂)ₐ-NH₂ group where a equals 1 to 10, preferably 1 to 6, and most preferably 1 to 4;
the R³ groups within the structures, independently from one another, can be the same or different and represent a hydrogen atom or a C₁ to C₂₀ hydrocarbon, preferably a C₁ to C₂₀ alkyl group or a C₆ to C₂₀ aryl-groups-containing radical, especially preferably a C₁ to C₁₀ alkyl group, or a C₆ to C₁₅ aryl-groups-containing radical, even more preferably a C₁ to C₄ alkyl group or a C₆ to C₁₀ aryl-groups-containing radical, wherein the methyl group and the phenyl group and the methyl group thereof are most preferred, wherein the C₁ to C₂₀ hydrocarbon can also contain heteroatoms, preferably oxygen or nitrogen;
the X groups represent an —A—NR⁴R⁵ or an —A—N+R⁴R⁵R⁶ group, wherein
   A stands for an at least divalent, preferably a divalent, C₁ to C₂₀ alkylene group, preferably C₁ to C₁₅ alkylene group, and especially preferably C₁ to C₁₀ alkylene group, which can also contain heteroatoms;
   R⁴, R⁵, and R⁶, independently from one another, can be the same or different, and represent a hydrogen atom or a C₁ to C₂₅ hydrocarbon, preferably a C₁ to C₂₀ alkyl group or a C₆ to C₂₀ aryl-groups-containing radical, especially preferably a C₁ to C₁₀ alkyl group or a C₆ to C₁₅ aryl-groups-containing radical, even more preferably a C₁ to C₄ alkyl group or a C₆ to C₁₀ aryl-groups-containing radical, wherein the methyl group and the phenyl group and the methyl group thereof are most preferred,
   wherein the following X groups are especially preferred:
—(CH₂)₃-NH₂,
—(CH₂)₃-NH₃⁻,
—(CH₂)₃OCH₂CHOHCH₂NH₂,
—(CH₂)₃NHCH₂CH₂NH₂,
—(CH₂)₃-N(CH₂CH₂OH)₂ as well
—(CH₂)₃OCH₂CHOHCH₂N⁺(CH₃)₂R⁷, wherein R₇ can have a C₁ to C₂₂ alkyl group, preferably a C₁ to C₁₅ alkyl group, and most preferably a C₁ to C₁₀ alkyl group, which can also have OH groups;
   m is a number of from 1 to 10,000, preferably of from 1 to 5,000, even more preferably of from 1 to 1,000, and most preferably of from 1 to 100;
   n is a number of from 1 to 500, preferably of from 1 to 250, even more preferably of from 1 to 100, and most preferably of from 1 to 50; and
   o is a number of from 0 to 200, preferably of from 5 to 150, even more preferably of from 10 to 100, and most preferably of from 20 to 50.

The individual structural units —OSiR₃R₃— and —OSiXR₃— can be statistically distributed within structures I and III but can also be arranged in blocks. Also conceivable as an organo-modified silicone is a linear, preferably nonvolatile amino-functional silicone, which is obtainable by combining structures I and II, with which thus the X groups can be located both terminally and laterally.

Examples of possible amino-functional silicones that can be used as an organo-modified silicone in the compositions according to the invention are the polydimethylsiloxanes known with the INCI designation (INCI=International Nomenclature of Cosmetic Ingredients) “Amodimethicone” or “PEG-x Amodimethicone” with terminal or lateral aminoalkyl groups, wherein x indicates the degree of ethoxylation, which is preferably between 2 and 10. Examples of Amodimethicone are the commercially available product known as Dow Corning® 939 Cationic Emulsion or the products known as Dow Corning® 949 Cationic Emulsion, Silicone XF 49-703, Silicone XF 42-B1989, or Mirasil ADM-E. Examples of an amino-functional silicone with a quaternary amino group are the dimethylsiloxanes with two terminal aminoalkyl groups with the INCI designation Quaternium-80. Other examples of amino-functional silicones with a quaternary amino group are the commercially available products Abil® Quat 3270, Abil® Quat 3272, or Abil® Quat 3274. Additional possible amino-functional silicones also include siloxane polyether amines such as Abil® Soft AF 100.

### C₃-C₂₂ Alcohol (Component B)

C₃-C₂₂ alcohols that are preferred according to the invention are monohydric or dihydric alcohols, wherein monohydric or dihydric alcohols are particularly preferred and dihydric alcohols are most preferred. Examples of preferred monohydric alcohols are butanol, pentanol, hexanol, heptanol, octanol, nonanol, decanol, myristyl alcohol, or behenyl alcohol. Particularly preferred dihydric alcohols are 1,2-propanediol, 1,3-propanediol, 1,2-butanediol, 1,3-butanediol, or 1,4-butanediol, wherein 1,3-butanediol is most preferred. Also conceivable are mixtures of at least two of the aforementioned monohydric or dihydric C₃-C₂₂ alcohols.

### Cationic Surfactant (Component C)

Any surfactants with at least one cationic, functional group can be used as cationic surfactants, wherein surfactants with a quaternary nitrogen group as a cationic, functional group are particularly preferred.

Examples of cationic surfactants with a quaternary nitrogen group are long-chain ammonium compounds as they are known with the CTFA designations (CTFA=Cosmetic, Toiletry and Fragrance Association). This includes, for example, alkyl trimethyl ammonium salts or dialkyl dimethyl ammonium salts with C₈ to C₂₂ alkyl groups. Additional examples of cationic surfactants are compounds with the structure

N⁺R⁸R⁹R¹⁰R¹¹⁻

wherein R⁸ to R¹¹, independently of one another, mean aliphatic groups, aromatic groups, alkoxy groups, polyoxyalkylene groups, alkylamido groups, hydroxyalkyl groups, aryl groups, or alkaryl groups each with 1 to 22 C atoms, wherein at least one of the radicals R⁸ to R¹¹ has at least 8 C atoms and X⁻ represents a cosmetically harmless anion, e.g. a halogen, acetate, phosphate, nitrate, or alkylsulfate, preferably a chloride. In addition to the carbon atoms and hydrogen atoms, the aliphatic groups can also contain cross-links or other groups such as, for example, other amino groups. Examples of suitable cationic surfactants are the chlorides or bromides of alkyl dimethyl benzyl ammonium salts, alkyl trimethyl ammonium salts, e.g. cetyltrimethyl ammonium chloride or bromide, tetradecyltrimethyl ammonium chloride or bromide, alkyl dimethyl hydroxyethyl ammonium chlorides or bromides, the dialkyl dimethyl ammonium chlorides or bromides, alkylpyridinium salts, e.g. lauryl or cetylpyridinium chloride, alkylamido ethyl trimethyl ammonium ether sulfates, as well as compounds with cationic character such as amine oxides, for example alkyl methyl amine oxides or alkyl amino ethyl dimethyl amine oxide. Cetryltrimethyl ammonium chloride is particularly preferred.

Suitable cationic surfactants are particularly also the so-called ester quats, e.g. C₈ to C₁₈ alkylesters of betaine, for example palmityl betaine chloride. The production of ester quats is described in European patent applications EP 0 284 036 A2, EP 0 669 391 A2, and EP 0 550 361 A1; the use of these ester quats for the care of keratin and other fibers is known from international patent application WO 96/03970 A1 and European patent applications EP 0 636 356 A1, EP 0 689 532 A1, and EP 0 614 349 A1. The aforementioned printed publications are listed here as references and the disclosure of the printed publications is considered a part of the disclosure of the present invention with respect to the ester quats. Ester quats are available, for example, under the trade names REWOQUAT® WE 18, REWOQUAT® WE 38 DPG (Witco Surfactants GmbH), Stepantex® GS 90 (Stepan), Armocare® VGH-70 (Alkzo Nobel), or Dehyquat®L-80 (Henlkel).

In addition to the aforementioned cationic surfactants, there are additional suitable cationic or amine-substituted surfactants such as those of the formula

R¹³—NH—(CH₂)ₙ—NR¹³R¹⁴

or the formula

R¹²—NH—(CH₂)ₙ—N⁺R¹³R¹⁴R¹⁵X⁻

wherein R¹³ is an acyl or an alkyl radical with 8 to 24 C atoms, which can be branched or unbranched, saturated or unsaturated, wherein the acyl and/or the alkyl radical can contain one or more OH groups, R¹³, R¹⁴, and R¹⁵, independently from one another, are hydrogen, alkyl radicals, or alkoxyalkyl radicals with 1 to 6 C atoms, which can be the same or different, saturated or unsaturated, or substituted with one or more hydroxy groups, X- is an anion, particularly a halogenide ion or a compound of general Formula RSO₃⁻, wherein R means saturated or unsaturated alkyl radicals with 1 to 4 C atoms, and n is a whole number between 1 and 10, or preferably of from 2 to 5.

### Volatile Silicone Oils (Component D)

Any silicone oils whose vapor pressure is greater than 0.1 mbar, preferably greater than 0.5 mbar, especially preferably greater than 1 mbar, even more preferably greater than 10 mbar, further preferably greater than 50 mbar, and most preferably greater than 100 mbar at a temperature of 21° C. can be used as the volatile silicone oils.

Volatile silicone oils preferred according to the present invention are silicones that are liquid and volatile at room temperature such as cyclic dimethyl siloxanes with 3 to 8, preferably 4 to 6 Si atoms, particularly cyclotetradimethylsiloxane, cyclopentadimethylsiloxane, or cyclohexadimethylsiloxane. Additional preferred volatile silicone oils are dimethylsiloxane/methylalkylsiloxane cyclo-copolymers, for example Silicone FZ 3109 from Union Carbide, which is a dimethylsiloxane/methyloctyl cyclo-copolymer. Linear volatile silicone oils that are preferred according to the present invention have 2 to 9 Si atoms. Preferred linear, volatile silicone oils include, for example, hexamethyldisiloxane or alkyltrisiloxanes such as hexylheptamethyltrisiloxane or octylheptamethyltrisiloxane. Additional preferred volatile silicone oils are low-viscosity polydimethylsiloxanes with a viscosity determined according to the test method described herein of less than 1,000 mPa.s, preferably less than 500 mPa.s, even more preferably less than 100 mPa.s and most preferably less than 50 mPa.s, as well as further preferably in a range of from 0.1 to 50 mPa.s and even further preferably of from 0.25 to 25 mPa.s. Preferred low-viscosity silicone oils include, for example, the commercially available product Dow Corning® 200 Fluid 5 CST and Dow Corning® 345 Fluid.

### Additional Additives (Component F)

Additional additives that are different from components a to e include other active ingredients, preferably active hair care ingredients.

An additional active hair care ingredient is preferably an amido amine or a quaternized amido amine of the aforementioned formulas

R¹²—NH—(CH₂)ₙ—NR¹³R¹⁴

or the formula

R¹²—NH—(CH₂)ₙ—N⁺R¹³R¹⁴R¹⁵X⁻

wherein R¹² is a branched or unbranched, saturated or unsaturated acyl radical with 8 to 24 C atoms, which can contain at least one OH group. Also preferred are those amines and/or quaternized amines in which at least one of the radicals R¹³, R¹⁴, and R¹⁵ mean a radical according to the general formula CH2CH2OR16, wherein R¹⁶ can mean alkyl radicals with 1 to 4 C atoms, hydroxyethyl, or H.

If the active hair care ingredient is a quaternized amido amine, this amido amine has the function of both a cationic surfactant (component a) and an additive (component f). These quaternized amido amines should, at the same time, also be considered preferred cationic surfactants.

Suitable amines or amido amines, which, if necessary, can be quaternized, are those, for example, with the INCI designations Ricinoleamidopropyl Betaine, Ricinoleamidopropyl Dimethylamine, Ricinoleamidopropyl Dimethyl Lactate, Ricinoleamidopropyl Ethyldimonium Ethosulfate, Ricinoleamidopropyltrimonium Chloride, Ricinoleamidopropyltrimonium Methosulfate, Cocamidopropyl Betaine, Cocamidopropyl Dimethylamine, Cocamidopropyl Ethyldimonium Ethosulfate, Cocamidopropyltrimonium Chlorid, Behenamidopropyl Dimethylamine, Isostearylamidopropyl Dimethylamine, Stearylamidopropyl Dimethylamin, Quaternium-33, and Undecylenamidopropyltrimonium Methosulfat.

Additional additives include, for example, thickeners to adjust viscosity such as starch or cellulose derivatives, glycol ethers, or glycols such as glycerin, keto carboxylic acids, and/or physiologically compatible salts thereof, sun-protective agents, and/or UV absorbers, preservatives, antioxidants (for example, tocopherols or esters thereof), dyes, as well as perfumes.

Furthermore, reference can be made to additives contained in hair care agents that are known to a person skilled in art in the present literature, which describes the general composition of hair care agents, for example SCHRADER, K., Fundamentals and Formulations of Cosmetics, 2nd edition, 1989, pages 728-737 or DOMSCH, A., Cosmetic Preparations, Verlag für chemische Industrie [publishing company] (H. Ziolkowsky, Ed.), 4th edition, vol. 2, pages 212-230, 1992, or JOHNSON, D. H. (publisher), Hair and Hair Care, New York, 1997, pages 65-104.

Additives that can be contained in the composition according to the present invention are, for example, cationic or water-soluble, nonionic polymers, vegetable and mineral fats and oils, perfume oils, dyes that are typical in hair cosmetics, additional hair-conditioning agents such as synthetic and natural phospholipids, quaternary derivatives of starch or cellulose, proteins or protein derivatives such as, for example, protein hydrolysates (for example, collagen, keratin, silk protein, or wheat protein hydrolysates), amino acids (for example, histidine, glycine, alanine, serine, threonine, arginine, cysteine, and derivatives thereof, for example of the fatty acid condensation products), plant extracts, vitamins, or provitamins (for example, biotin, vitamin C, D-panthenol of the derivatives thereof), allantoin, chitosan, viscosity-regulating materials such as fatty acid alkanol amides, alkoxylated esters of polyols (for example, glycerol, sorbitol, fructose or glucose, anti-dandruff agents, and inorganic or organic acids (for example, acetic acid, lactic acid, citric acid, glycolic acid, malic acid, phosphoric acid).

It is further preferred according to the invention if the composition has a viscosity determined according to the test method described herein in a range of from 0.1 to 1,000 mPa.s, especially preferably in a range of from 0.5 to 500 mPa.s, and most preferably in a range of from 0.7 to 50 mPa.s, as well as further preferably in a range of from 0.8 to 5 mPa.s, and further preferred in a range of from 0.9 to 2.5 mPa.s. Furthermore, it is preferred according to another embodiment of the composition according to the present invention if the viscosity of the composition determined according to the test method described herein is less than 100 mPa.s, especially preferably less than 50 mPa.s, further preferably less than 25 mPa.s, and most preferably less than 10 mPa.s.

The composition according to the invention can be preferably used as a cosmetic hair care agent and can be used as a spray or foam. In doing so, the composition can be used for all known cosmetic applications that typically involve the hair. This can be, for example, in the form of a spray hair-repair agent, a foam hair-repair agent, a rinse, or a treatment. The hair care agent can also be used as a pretreatment agent before hair coloring or before permanent wave treatment in order to condition the hair during the damaging treatment as well. Furthermore, the composition can also be used as a pretreatment agent before the application of a hair-repair cream to intensify the conditioning effect of the hair-repair cream.

In addition, the composition according to the present invention can also be used as a two-phase product. Two-phase products are, for example, described in DE 197 03 475. If the composition according to the present invention is used as a two-phase product, the composition according to the present invention thus forms a first hydrophilic phase whereas preferably a volatile silicone compound or a volatile hydrocarbon, but preferably a volatile hydrocarbon, is used as the second lipophilic phase, which is preferably visibly different from the first phase. Preferred components of the lipophilic phase, as well as preferred quantity ratios of the hydrophilic and lipophilic phase if the composition according to the present invention is used as a two-phase product, are those components and quantity ratios that are disclosed in DE 197 03 475 A1. The disclosure of DE 197 03 475 A1 regarding this constitutes a part of the disclosure of the present invention.

With the process according to the present invention to produce a suitable composition, preferably as a hair care agent, containing
a) an organo-modified silicone;
b) a C₃-C₂₂ alcohol preferably different from component e);
c) if necessary, a cationic surfactant;
d) if necessary, a volatile silicone oil;
e) at least 30 wt %, preferably at least 40 wt %, especially preferably at least 50 wt %, and further preferably at least 60 wt %, based on the total weight of the composition in each case, ethanol or isopropanol, preferably ethanol; as well as
f) if necessary, an additive that is different from components a to e;
   components a to f are placed in contact with one another by mixing.

Preferred components a to f as well as preferred quantities of components a to f are those components and/or quantities that were indicated at the start, within the context of the composition according to the present invention.

The individual components are placed in contact with one another preferably at room temperature, wherein a person skilled in the art can obtain the most advantageous sequence of mixing of the individual components through routine pretests.

With the process according to the present invention to treat hair, the hair is placed into contact with the composition described at the beginning or with the composition obtainable through the process according to the present invention described previously, wherein if the composition according to the present invention is used as a two-phase product, this two-phase product is preferably shaken before application to the hair in order to effect a mixing of the two phases.

The composition according to the present invention can either remain in the hair after application to the dry, damp, or wet hair, or it can be rinsed out after a suitable action period. The action periods depend on the type of hair as well as the temperature, wherein the temperature is preferably in a range of from 20 to 50° C. As a general guideline however, action periods between 0.5 and 60 minutes, particularly between 1 and 40 minutes, or preferably between 5.0 and 20 minutes can be assumed, wherein the repair effect can be accelerated by adding heat. Once the action period is over, the hair can be rinsed with water.

The composition according to the present invention can, depending on the adjusted viscosity, advantageously be applied or sprayed onto the dry or damp hair with the aid of typical pumps and dispensing aids, for example, as an aerosol. Suitable spray or foaming devices are known to a person skilled in the art; these devices can be operated with a propellant or with the help of mechanically generated pressure generation. The application preferably occurs after the hair has been washed.

If the composition according to the present invention is supposed to be applied with the aid of a propellant, particularly in the form of a spray or foam, it can be filled into a suitable pressurized container, particularly into an aerosol can, along with the typical propellants in the typical ratios or quantities. An example of a ratio of composition according to the present invention to propellant would be 92:8. The inner pressure depends on the type of container used. At a temperature of 20° C., pressure in a range of from 1.5 to 8.0 bar can be used as a guideline. Suitable propellants are, for example, lower alkanes, such as n-butane, i-butane, and propane, dimethylethers, as well as gaseous propellants that are present, such as N₂, N₂O, or CO₂, as well as mixtures of the aforementioned propellants.

Mechanical spraying or foaming devices are preferably understood to mean those devices that enable spraying or foaming without the use of a propellent. Suitable mechanical spray or foaming devices can include, for example, a pump or an elastically deformable container with a spray valve in which the composition according to the present invention is filled. If the containers are filled under pressure, the composition according to the present invention can be dispensed continuously or in metered quantities, after the spray or foaming valve has been opened. Alternatively, the pressure needed to dispense the composition according to the present invention can be created through reversible mechanical deformation of the container.

The invention also relates to the use of the aforementioned composition or the composition obtainable through the aforementioned process for preferable cosmetic treatment of hair, wherein, in particular, the use of the composition as a rinse-off product is preferred.

Furthermore, the invention also relates to the use of the aforementioned composition or the composition obtainable through the aforementioned process to produce an agent for preferable cosmetic treatment of hair.

The invention will now be explained in more detail using examples.

### EXAMPLES

The components were mixed together at room temperature in the relative quantities indicated in the individual tables.

### Example 1

| Raw material | INCI/EU | percent by weight |
|---|---|---|
| Cetyl trimethylammonium chloride | Cetrimonium Chloride | 0.50 |
| Diquaternary polydimethylsiloxane | Quaternium-80 | 0.10 |
| Cyclopentadimethylsiloxane | Cyclomethicone | 8.00 |
| 1,3-Butanediol | Butylene Glycol | 1.00 |
| Ethanol | Denat. Alcohol | 85.00 |
| Perfume oil | Perfume | 0.30 |
| water, completely desalinated | Aqua | balance to 100 |
| Viscosity | 1 | mPas · s |

### Example 2

| Raw material | INCI/EU | percent by weight |
|---|---|---|
| Behenyl trimethyl ammonium chloride | Behentrimonium Chloride | 0.40 |
| Amino-functional polydimethylsiloxane | Amodimethicone | 0.20 |
| Low-viscosity polydimethylsiloxane | Dimethicone | 5.00 |
| 1,3-Butanediol | Butylene Clycol | 1.50 |
| Ethanol | Denat. Alcohol | 85.00 |
| Perfume oil | Perfume | 0.30 |
| water, completely desalinated | Aqua | balance to 100 |

### Example 3

| Raw material | INCI/EU | percent by weight |
|---|---|---|
| Dimethyl ditallow ammonium | Quaternium-18 | 0.40 |
| chloride | | |
| Amino-functional | Amodimethicone | 0.20 |
| polydimethylsiloxane | | |
| Cyclotetradimethylsiloxane | Cyclomethicone | 5.00 |
| 1,3-Butanediol | Butylene Clycol | 0.80 |
| Ethanol | Denat. Alcohol | 85.00 |
| Perfume oil | Perfume | 0.30 |
| water, completely desalinated | Aqua | balance to 100 |

### Example 4

| Raw material | INCI/EU | percent by weight |
|---|---|---|
| Stearamidopropyl dimethylamine | Stearamidopropyl | 0.60 |
| | Dimethylamine | |
| Diquaternary polydimethylsiloxane | Quaternium-80 | 0.25 |
| Cyclotetradimethylsiloxane | Cyclomethicone | 3.00 |
| 1,3-Butanediol | Butylene Glycol | 0.80 |
| Ethanol | Denat. Alcohol | 60.00 |
| Perfume oil | Perfume | 0.30 |
| water, completely desalinated | Aqua | balance to 100 |

The preparation from Example 1 was subjected to a half-side test in a hairstylist's assessment. In this process, the criteria
distribution capacity,
combability (damp),
feel (damp),
volume, and
harshness
   were assessed in comparison to a conventional hair-repair cream (a viscous emulsion based on a cationic surfactant and a fatty alcohol) on 5 different test subjects; the numbers indicate the number of test subjects on which the respective criterion was assessed and how it was assessed:

| | Better than hair- | The same as hair- | Worse than hair- |
|---|---|---|---|
| Criteria | repair cream | repair cream | repair cream |
| Spreadability | 5 | — | — |
| Combability | 2 | 1 | 2 |
| (damp) | | | |
| Hold when wet | 1 | 3 | 1 |
| Volume | 4 | 1 | — |
| Harshness | 4 | 1 | — |

The results clearly show that the composition according to the present invention not only has an improved distribution capacity on the hair as compared to a conventional hair-repair cream, but the stress of the dry hair is less (better) than it is when the hair-repair cream is used and the volume of the hair is greater (better), wherein the composition according to the present invention does not have any reduced care effect (combability and feel of the damp hair) as compared to the hair-repair cream.

### Test Methods

### Determining Viscosity

The viscosity was determined using the following device and under the following conditions:

Rheometer: Bohlin Rheometer CS

Measurement program: Yield Stress

Measurement system: DG 40/50

Shear rate: 50 s⁻¹

Delay time: 5 s

Integration time: 5 s

Temperature: 25° C.

### Hairstylist's Assessment in the Half-Side Test

The method is based on so-called half-side comparisons on multiple test subjects (models); the comparisons are made by a trained hairstylist. In a half-side comparison, the scalp hair is divided up into two sections from the forehead to the neck. A random section remains untreated or receives a comparison product while the test product is applied to the other section.

The models' hair, which is of varying lengths, is then washed twice with a chemically neutral shampoo. After being washed, the hair is towel-dried lightly. The towel-dried scalp hair is divided up into two sections by separating the hair down the center of the head from the forehead to the neck. A random section receives the composition according to the invention; the same quantity by weight of hair-repair cream is applied to the other section.

The hairstylist providing the assessment then evaluates how easy it is to distribute the product being assessed in the towel-dried hair by distributing the relevant product in the hair with both hands.

After the action period, the product is rinsed out with careful consideration of maintaining the two separate sections (the sections cannot mix together).

Then, as an important hair care criterion, the feel of the damp hair is assessed, by the hairstylist providing the evaluation, by running the fingers through the hair from the roots to the tips with slight pressure of the fingers. If the fingers move through the hair without any resistance or with less resistance than [through the] hair of the half side that has received no product or that has been treated with a comparison product, this is characterized as an improved/smoother feel.

Subsequently, the combability of the wet hair is assessed by the hairstylist providing the evaluation by combing both sides with a hard rubber comb starting at the forehead and ending at the neck, wherein care needs to be taken to ensure that the comb is guided on the scalp so that no difference in the angle of the comb results. If the hair can be combed without any resistance or with less resistance than the hair without any product or that treated on one side with a comparison product, this is characterized as easy, good, improved combability.

After the hair is dried completely, the hair volume is assessed by the hairstylist providing the evaluation by visually ascertaining the larger hair volume. The evaluator compares both sections at a distance of one meter, while standing behind the model. The hair is then combed on both sides with a comb and the volume is subsequently assessed, also at a distance of one meter, as the model performs a headshake twice.

Subsequently, they stress on the hair is assessed by the hairstylist providing the evaluation by running the fingers through the hair from the roots to the tips with slight pressure of the fingers. If there is no film or less film remaining on the fingers than there is with the other side without any product or treated with a comparison product, this is defined as no or less stress.

The assessment of the technical hairstylist criteria mentioned is implemented as described and can be reproduced at any time if the guidelines are maintained, whereby an objective product comparison is possible.

The half-side comparison provides relevant results as long as the result for a tested criterion is not equal to “the same.”

What is claimed is:

1. A composition containing as components
a) an organo-modified silicone;
b) if necessary, a C₃-C₂₂alcohol;
c) if necessary, a cationic surfactant;
d) if necessary, a volatile silicone oil;
e) at least 30 wt % ethanol, in relation to the total weight of the composition; as well as
f) if necessary, additional additives that are different from components a to e,
wherein the total quantity of components a to f is 100 wt %.

2. The composition according to claim 1, wherein the composition contains, in addition to component e, the following:
0.01 to 15 wt % of component a,
0.01 to 40 wt % of component b,
0.01 to 15 wt % of component c,
0.01 to 40 wt % of component d, as well as
0.01 to 15 wt % of component f.

3. The composition according to claim 1, wherein the composition has a viscosity determined according to the test method described herein in a range of from 0.1 to 1,000 mPa.s.

4. The composition according to claim 1, wherein the organo-modified silicone has an average molecular weight in a range of from 100 to 1,000,000.

5. The composition according to claim 1, wherein the organo-modified silicone is a nonvolatile, nitrogen-containing silicone.

6. The composition according to claim 1, wherein the nitrogen-containing silicone has an amino-functional silicone with an amine portion (meq/g) in a range of from 0.01 to 3.0.

7. The composition according to claim 6, wherein the nitrogen-containing silicone is a linear, amino-functional silicone of Structure I 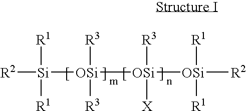
or a linear, amino-functional silicone of Structure II 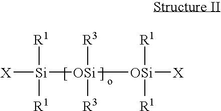
or a cyclic, amino-functional silicone of Structure III 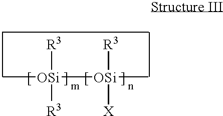
wherein
the R¹ groups within the structures, independently of one another, can be the same or different and represent a hydrogen atom, a C₁ to C₁₀ alkyl group, a radical containing aryl groups, a hydroxy group, a C₂ to C₁₀ alkoxy group, or a C₁ to C₁₀ acetoxy group;
the R² groups within the structures, independently of one another, can be the same or different and represent a hydrogen atom, a C₁ to C₁₀ alkyl group, a radical containing aryl groups, a hydroxy group, a C₁ to C₁₀ alkoxy group, a C₂ to C₁₀ acetoxy group, or a —(CH₂)ₐ-NH₂ group where a equals 1 to 6;
the R3 groups within the structures, independently from one another, can be the same or different and represent a hydrogen atom or a C₁ to C₂₀ hydrocarbon, which can contain heteroatoms;
the X groups represent an —A—NR⁴R⁵ or an —A—N⁺R⁴R⁵R⁶ group, wherein
A stands for an at least divalent C₁ to C₂₀ alkylene group, which can also contain heteroatoms;
R⁴, R⁵ and R⁶, independently from one another, can be the same or different and represent a hydrogen atom or a C₁ to C₂₂ hydrocarbon;
m is a number of from 1 to 10,000;
n is a number of from 1 to 500; and
o is a number of from 0 to 200.

8. The composition according to claim 1, wherein the C₃-C₂₂ alcohol is a dihydric alcohol.

9. The composition according to claim 8, wherein the C₃-C₂₂ alcohol is selected from the group consisting of 1,2-propanediol and 1,3-butanediol.

10. A process to manufacture a composition containing
a) an organo-modified silicone;
b) a C₃-C₂₂ alcohol;
c) if necessary, a cationic surfactant;
d) if necessary, a volatile silicone oil;
e) at least 30 wt % of a solvent that is different from components a to d;
f) as well as, if necessary, an additive that is different from components a to e;
wherein components a to f are placed in contact with one another by mixing.

11. A composition that is obtainable through a process according to claim 10.

12. A process to treat hair, wherein the hair is placed in contact with a composition according to claim 1.

13. Use of the composition according to claim 1 to treat hair.

14. Use of the composition according to claim 1 produce an agent to treat hair.
